(19) United States
(12) Patent Application Publication
(10) Pub. No.: US 2011/0021993 A1
(43) Pub. Date: Jan. 27, 2011

(54) MINIATURE DISPOSABLE OR PARTIALLY REUSABLE DOSING PUMP

(75) Inventors: Avraham Bar-Haim, Ma'alot (IL); Thomas Philip, Rehovot (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: GAIA MED LTD., Carmei Yosef (IL)

(21) Appl. No.: 12/922,699

(22) PCT Filed: Mar. 19, 2009
(86) PCT No.: PCT/IL09/00312

§ 371 Date: Sep. 15, 2010

(30) Foreign Application Priority Data
Mar. 20, 2008 (IL) 190335

(51) Int. Cl. A61M 5/148; A61M 5/142

(52) U.S. Cl. 604/153; 604/151

(57) ABSTRACT

The invention is a dosing pump that can be worn on the body of a patient for subcutaneously delivering liquid with centi-micro liter accuracy to his/her body. The dosing pump comprises a pump unit, an internal control unit; and a reservoir containing the liquid. The pump unit comprises a pump block (12) comprising a pump chamber (118) with a pump diaphragm (107) stretched across its entrance and a pump pin cylinder (110) extending from the exterior of the pump block to the entrance to the pump chamber; a pump pin (20) that is located in the pump pin cylinder and a motor unit comprising a motor and gear system. When the motor is activated, the rotational motion of the motor and the gears in the gear system is transformed into cyclic back and forth linear motion of the pump pin in the pin cylinder pumping the liquid from the reservoir into the patient. Unlike standard piston pumps, the pump diaphragm is not attached to the pump pin and the only force required to be exerted by the motor is to move the pin back and forth and not to pull the diaphragm back, i.e. in the present invention the force required to create the suction is provided by the internal energy stored in the stretched diaphragm and not by the mechanism that moves the piston.

### FIELD OF THE INVENTION

The present invention relates to the field of medical devices. Specifically the present invention relates to dosing pumps for injecting small, precisely measured doses of liquid into a patient's body. More specifically the present invention relates to miniature disposable or partially reusable dosing pumps that can be carried by the patient or attached to his body as he pursues his normal daily routine.

### BACKGROUND OF THE INVENTION

A wide range of abnormal medical conditions are caused by either a lack or an excess of specific chemicals, e.g. hormones or vitamins, in the body.

Patients suffering from diabetes mellitus are generally characterized by a relative or complete lack of insulin secretion by the beta cells of the pancreas. Insulin acts to regulate the metabolism of glucose, lowering blood glucose levels and promoting transport and entry of glucose into the muscle cells and other tissues. Inadequate secretion of insulin progressively might result in complications such as eye disease, kidney disease, heart disease, and nerve damage.

These complications can be significantly reduced by frequent monitoring and maintenance of blood glucose levels. There are currently two main approaches for daily insulin therapy: The first approach includes use of syringes and insulin injection pens, that are user-friendly and relatively cheap, but require a needle prick at each injection; the second approach is infusion pump therapy, which entails the purchase of a relatively expensive device, which is one of the principal obstacles to this kind of therapy. Although more complex than syringes and pens, a pump offers the advantages of continuous infusion of insulin, precision in dosing and optionally programmable delivery profiles and user actuated bolus infusions in connection with meals.

Modern infusion devices appeared with the development of the microelectromechanical systems (MEMS) technology, which aims to provide low cost and high performance medical devices. MEMS are microscale systems which involve both electronic and non-electronic elements and perform functions that include sensing, processing, actuation, display and control with high functionality, precision and performance. MEMS drug delivery systems offer better drug therapy which allows accurate dosing with more efficiency and effectiveness. Applying MEMS to drug delivery by using biocapsules, microneedles, micropumps and microreservoirs offer a less invasive therapy and improved the quality of life of diabetic patients.

A micropump is a major component of MEMS drug delivery systems. In general, high volumetric flow rate and high resolution are both of great importance when designing such microelements. The flow rate is usually directly correlated to the pressure generated by the actuator but other essential concerns should be taken into account like reliability, biocompatibility and power consumption. At the present time, many different types of micropumps have been designed to handle small and precise volumes of chemical or biological solutions. Technically, micropumps may be categorized into two groups: mechanical pumps, which include a physical actuator or mechanism to perform the pumping function; and non-mechanical pumps, which have to transform available non-mechanical energy into kinetic momentum so that the liquid in micro channels can be driven. Mechanical micropumps are currently the most popular and include several microelements such as microchannels, microchambers, microvalves, and microactuators that are responsible of the liquid motion either by direct interaction with the liquid or by indirect interaction, usually using a diaphragm. These pumps may be classified according to the force driving the actuator.

U.S. Pat. No. 4,552,561 discloses an infusion device based on an osmotic pump that operates by causing water to pass through a semi-permeable membrane to displace liquid from the insulin dispenser. One of the main problems with this configuration is that the flow rate of the osmotic pump varies with temperature. A change in body or external temperature could have the undesirable effect of changing the flow rate of the medicament.

U.S. Pat. No. 5,205,819 discloses a piezoelectric micropump comprising a pressure chamber which is partly bounded by a membrane. The membrane is actuated by a piezoelectric component, consisting of piezoelectric crystals that create an axial deformation under application of a specific voltage. Building such a pump necessitates complex manufacturing steps including binding of the piezoelectric elements to the membrane.

U.S. Pat. No. 7,033,148 discloses an electromagnetic micropump, comprising a magnetic actuator assembly, a flexible membrane, a housing defining a chamber, and a plurality of valves. The magnetic actuator assembly comprises a coil and a permanent magnet for deflecting the membrane to effect pumping of the liquid. A disadvantage of the electromagnetic actuator is generally the relatively large volume occupied by the coil, increasing considerably the size of the overall device.

U.S. Pat. No. 6,520,753 discloses a thermopneumatic micropump including a chamber plate with connected pumping chambers and a pumping structure. The pumping structure includes a flexible membrane, portions of which may be inflated into associated pumping chambers. A working fluid is heated by microheating elements in cavities below the flexible membrane portions, in order to inflate the membrane and create liquid motion. A complicated structure and a slow response are the main shortcomings of thermopneumatic pumps.

U.S. Pat. No. 6,729,856 discloses a device for electrostatically pumping liquids. Electrostatic forces are used to move diaphragms in one direction, while elastic and/or other restorative forces are used to move the diaphragms back to their original un-activated positions. The major shortcomings of this type of pump are the relatively complicated microstructure and the high applied voltage required.

U.S. Pat. No. 6,299,419 discloses a piston micropump, where a reciprocating diaphragm pump is actuated by a piston-cylinder unit, which is sealed by hydrodynamic sealing. In such micropumps, diaphragms endure very extensive deformations due to the direct coupling with the piston, therefore decreasing the device's shelf-life.

Addressing the above problems, several attempts have been made to provide insulin infusion devices that are low in cost and convenient to use. Some of these devices are intended to be partially or entirely disposable and may provide many of the advantages associated with an infusion pump without the attendant cost and inconveniencies, e.g. the pump may be pre-filled thus avoiding the need for filling or refilling a drug reservoir.

Although it can be expected that the above described class of fully or partly disposable infusion devices can be manufactured considerably cheaper than the traditional durable infusion pump, they are still believed to be too expensive to be used as a real alternative to traditional infusion pumps and attached infusion sets for use on an every-day basis. Clearly, therefore, there is a need for a programmable and adjustable infusion system that is precise and reliable and can offer clinicians and patients a small, low cost, light-weight, easy-to-use alternative for delivery of insulin to the patient. The device should be economical to manufacture and capable of accurately delivering quantities of liquid in the centimicro-liter range as and when required.

It is therefore an object of this invention to present a small light-weight inexpensive insulin pump that provides convenience of use and centimicro-liter treatment control.

It is another object of the present invention to provide an insulin pump that can be attached directly to the body of the patient and controlled from a standard computing device using a standard operating system.

It is another object of the present invention to provide the pump of the invention in embodiments that are partially reusable or totally disposable after a single use.

Further purposes and advantages of this invention will appear as the description proceeds.

### SUMMARY OF THE INVENTION

The invention is a dosing pump that can be worn on the body of a patient for subcutaneously delivering liquid with centi-micro liter accuracy to his/her body. The dosing pump comprises:
A. a pump unit comprising:
   i) a pump block comprising:
      a.) a pump chamber with a pump diaphragm stretched across its entrance;
      b.) a pump pin cylinder extending from the exterior of the pump block to the entrance to the pump chamber;
      c.) an inlet chamber with an inlet diaphragm stretched across its entrance;
      d.) an outlet chamber with an outlet diaphragm stretched across its entrance; and
      e.) several channels to provide fluid communication between the pump, inlet, and outlet chambers and the outside of the pump block;
   ii) a pump pin comprising a rounded distal end that is located in the pump pin cylinder;
   iii) a motor unit comprising:
      a.) a motor; and
      b.) a gear system comprising a plurality of gears arranged to transfer the rotary motion of the shaft of the motor to an output axle coupled to the pump pin;
B. an internal control unit; and
C. a reservoir containing the liquid.

When the motor is activated, the rotational motion of the motor and the gears in the gear system is transformed into cyclic back and forth linear motion of the pump pin in the pin cylinder. As the pump pin is pushed forward, its rounded distal end pushes against the pump diaphragm causing the pump diaphragm to stretch and move forward, reducing the volume of the pump chamber, increasing the pressure on liquid located in the pump chamber and causing the outlet diaphragm to stretch into the outlet chamber. This allows the liquid to flow out of the pump block through the outlet chamber. When the pump pin is pulled backward, the internal forces created in the pump diaphragm by stretching it will act to return the pump diaphragm to its minimum energy position, thereby increasing the volume of the pump chamber, reducing the pressure in the pump chamber below atmospheric pressure and causing the inlet diaphragm to stretch into the inlet chamber. This allows the liquid to be sucked out of the reservoir via the inlet chamber into the pump chamber.

In the dosing pump of the invention, as the pump pin is moved forward, the pump diaphragm is stretched and its center is moved forward a distance that is greater than the diameter of the pump diaphragm.

The dosing pump of the invention can be used to deliver insulin to the body of a patient.

In embodiments of the dosing pump the output axle is coupled to the pump pin by means of an eccentric pin that is fixedly attached to the output axle and fits into a slot at the proximal end of the pump pin. In other embodiments the output axle is coupled to the pump pin by means of a pinion gear fixedly attached to the output axle, wherein the teeth of the pinion gear mesh with the teeth of a rack gear that is an integral part of the pump pin.

In embodiments of the dosing pump of the invention the pump pin, motor, gear system, and the pump diaphragm are strong enough to enable a force of at least five atmospheres to be exerted on the liquid in the pump chamber when the pump pin pushes against the pump diaphragm. In embodiments of the dosing pump, as the pump pin is pulled backwards, the energy stored in the pump diaphragm is released as the pump diaphragm returns to its unstretched state, thereby exerting a force of about two bars as the pump diaphragm contracts.

Embodiments of the dosing pump comprise a pressure sensor diaphragm one side of which is in fluid communication with the outlet chamber when liquid flows through the outlet chamber. When liquid is forced through the outlet chamber pressure, the pressure sensor diaphragm is caused to move relative to a pressure sensor, which measures parameters that can be translated into pressure measurements. The pressure sensor can be chosen from the following: an optical sensor; a conductive silicone membrane, which changes electrical conductivity when stretched; a strain gauge buried in the pressure sensor; and an ultrasound sensor. The output of the pressure sensor provides an indication that the dosing pump is operating properly and issues a real time warning if a problem with the flow of liquid from the reservoir to the body of the patient is detected. The problem can be one or more of the following: a blockage in the fluid path from the reservoir to the body of the patient, a leak in the fluid path, air in the fluid path, or a non-constant supply of liquid from the reservoir.

In embodiments of the dosing pump the motor turns in one direction only and the gear system comprises a sensor to measure the exact instant when the direction of linear motion of the pump pin changes. In other embodiments the direction in which the motor turns is reversed by the internal control unit based on the magnitude of the current drawn by the motor. Embodiments of the gear system comprise a sensor to measure each rotation of the shaft of the motor.

Embodiments of the dosing pump are entirely disposable after the reservoir is emptied once. In these embodiments the reservoir can be a collapsible elastomeric bladder.

Embodiments of the dosing pump of the invention can be attached directly to an infusion patch. These embodiments can comprise a quick release mechanism allowing the dosing pump to be easily temporarily disconnected from the infusion patch.

The dosing pump of the invention can comprise a button for administering a pre-determined bolus dose. Input/output to the internal control unit of the dosing pump can be from a hand held remote control unit using an RFID or Bluetooth connection. The hand held remote control unit can be a standard Palm-like device or a mobile phone.

Embodiments of the dosing pump of the invention are partially reusable. In some of the partially reusable embodiments, the non-reusable parts of the dosing pump comprise the reservoir, the pump pin, the pump block, and optionally, depending on its location in the dosing pump, the sensor diaphragm. In these embodiments the reservoir can be a standard 3 ml insulin pen cartridge.

In embodiments of the dosing pump of the invention the reservoir is a collapsible bladder made of an elastomeric material.

Embodiments of the dosing pump of the invention comprise an adhesive pad directly attached to the bottom surface of the pump for attaching the pump to the skin of a patient and a small hollow needle in liquid communication with the output channel of the pump block, which projects downward through the adhesive pad for penetrating the skin.

In some of the partially reusable embodiments of the dosing pump the non-reusable parts of the dosing pump are the pump block, the gear unit, the motor, the pressure sensor diaphragm, the reservoir, and the battery.

All the above and other characteristics and advantages of the invention will be further understood through the following illustrative and non-limitative description of preferred embodiments thereof, with reference to the appended drawings. In the drawings the same numerals are sometimes used to indicate the same elements in different drawings.

### BRIEF DESCRIPTION OF THE DRAWINGS

FIG. 1A and FIG. 1B are cross sectional views of one embodiment of the pump unit of the device of the invention showing the steady state and the compression stages respectively;

FIG. 2A and FIG. 2B are cross sectional views of a second embodiment of the pump unit of the device of the invention showing the suction and the compression stages respectively;

FIG. 3A, FIG. 3B, and FIG. 3C show different views of the pump diaphragm;

FIG. 4A, FIG. 4B, and FIG. 4C show different views of the inlet diaphragm;

FIG. 5A, FIG. 5B, and FIG. 5C show different views of the outlet diaphragm;

FIG. 6 shows the motor unit of the dosing pump of the invention;

FIG. 7 shows the gear train of the motor unit of the invention;

FIG. 8 illustrates the location and function of the sensors of the motor unit of the invention;

FIG. 9 and FIG. 10 are general views showing an embodiment of the disposable insulin pump of the invention and an infusion patch from the top and bottom sides respectively;

FIG. 11 is a top view of an embodiment of a disposable dosing/insulin pump according to the present invention with part of the cover removed to expose some of the internal components;

FIG. 12 is a cross-sectional view of the pump shown in FIG. 11 attached to an infusion patch;

FIG. 13 is a view of the disposable pump of the invention with part of the side sliced off to reveal the inside of the insulin reservoir/bladder and illustrate the method of filling the bladder with insulin;

FIG. 14 and FIG. 15 show an embodiment of a partially reusable dosing/insulin pump according to the present invention;

FIG. 16 and FIG. 17 show the top and bottom respectively of another embodiment of a partially reusable dosing/insulin pump according to the present invention;

FIG. 18 and FIG. 19 show the disposable part of a partially reusable dosing/insulin pump according to the present invention with part of the cover removed;

FIG. 20A and FIG. 20B are cross sectional views of the pump unit of the device of FIGS. 18 and 19 showing the suction and the compression stages respectively;

FIG. 21A and FIG. 21B show different views of the inlet diaphragm of the pump unit of FIGS. 20A and 20B;

FIG. 22A and FIG. 22B show different views of the outlet diaphragm of the pump unit of FIGS. 20A and 20B;

FIG. 23A and FIG. 23B respectively show the top and bottom of the reusable part of the partially reusable dosing/insulin pump whose disposable part is shown in FIGS. 18 and 19;

FIG. 24 shows the internal components of the reusable part of the partially reusable dosing/insulin pump shown in FIGS. 23A and 23B;

FIG. 25 shows an enlarged view of the motor and gear train of FIG. 24; and

FIG. 26 shows the reusable part of the partially reusable dosing/insulin pump inserted into the socket in the disposable part.

### DETAILED DESCRIPTION OF PREFERRED EMBODIMENTS

Although in the following description the invention is specifically referred to as an insulin pump, it is not the intention of the inventors to limit the use of the invention to the specific case of injection of insulin. The invention can be used as described, or with minor variations mutatis mutandis, as a dosing pump to inject small, precisely measured quantities of any other liquid into a patient. The inventors anticipate that the present invention will lead to many new treatment procedures, similar to those of insulin injection, which have not yet been contemplated or developed to the stage of widespread use because of the lack of availability of a dosing pump possessing the properties and capabilities of the pump of the present invention.

The invention is an insulin/dosing pump that is comprised of the following main elements:
A. Pump Unit;
B. Internal control unit; and
C. Reservoir.

The pump unit is the “heart” of the device. The pumping unit is comprised of two parts: a block 12 that has been machined to create and provide room for the elements that are involved in drawing the liquid out of the reservoir and in pushing it in the direction of the infusion set 50 and a motor unit 200 comprising a motor 202, gear train 204, and drive pin 20.

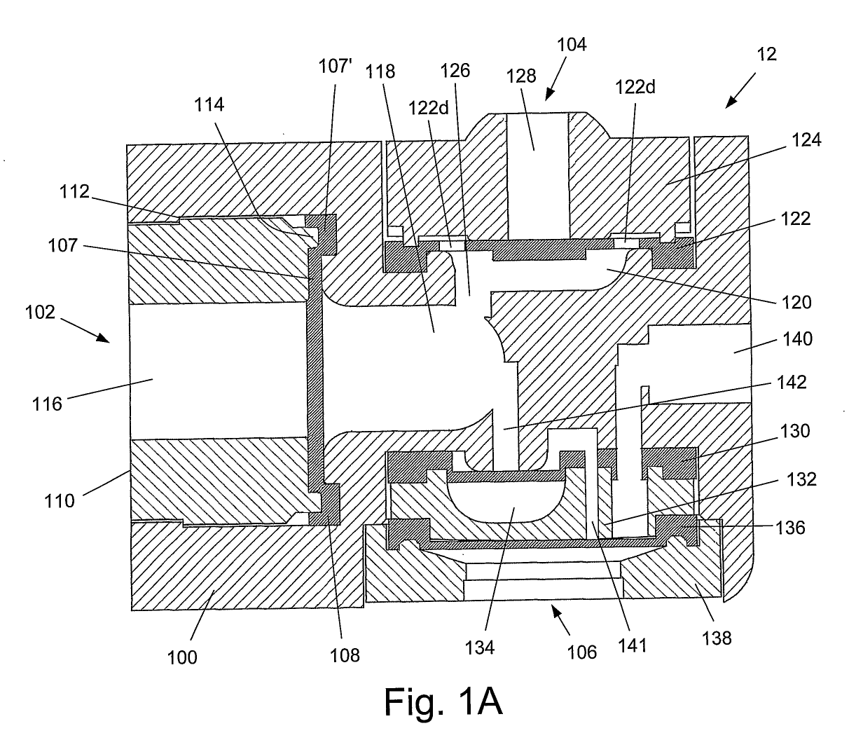

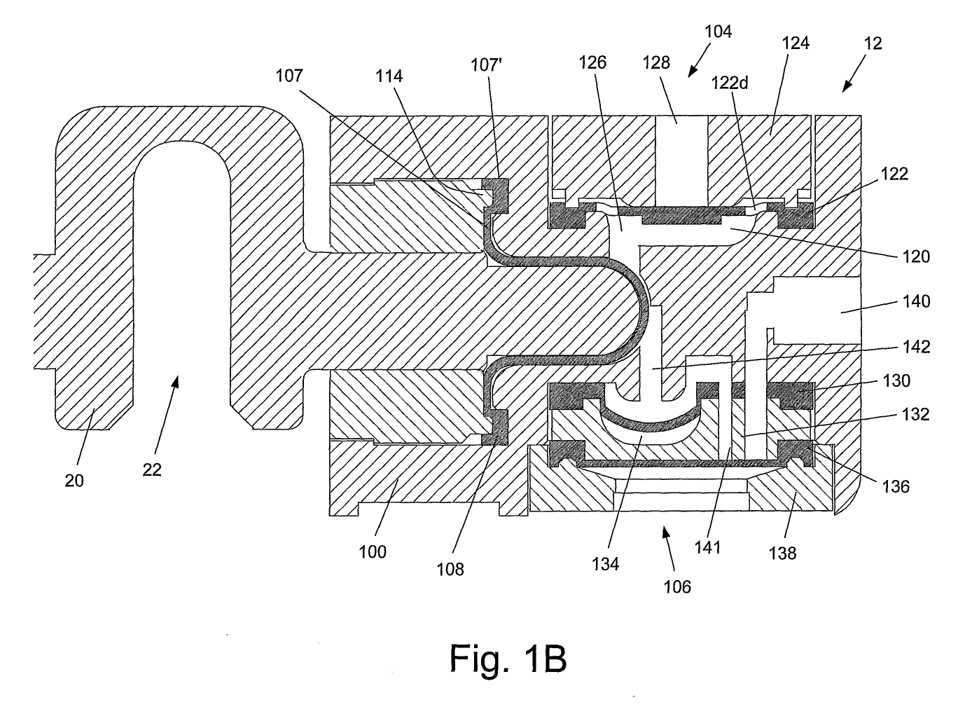

FIG. 1A and FIG. 1B are cross sectional views of one embodiment of block 12 showing the steady state and the compression stages respectively. Referring first to FIG. 1A, there can be seen three main cylindrical bores and several channels to provide liquid communication between the bores and the outside of the block that have been created in body 100 of block 12. The three bores are: pump bore 102, in which is located pump chamber 118; inlet bore 104, comprising inlet chamber 120; and outlet bore 106 comprising outlet chamber 134. The pump unit of the invention operates with a simple two stage cycle comprised of a suction stage in which the pressure in pump chamber 118 (see FIG. 1A) is reduced below atmospheric pressure, allowing liquid to be sucked out of the reservoir, thereby filling inlet chamber 120 and pump chamber 118 and a compression stage in which the liquid in pump chamber 118 is pushed out of the pump block 12 to the infusion set through outlet chamber 134.

Pump bore 102 is comprised of a distal section and a proximal section, which has a wider diameter than the distal section, thereby creating an annular step 107 at their interface. Around the outer circumference of this step is created a circular groove 107′ and together they form a seat for an appropriately shaped pump diaphragm 108. After the pump diaphragm 108 is placed on its seat, pump diaphragm cap 110 is slid into pump bore 102.

Pump diaphragm cap 110 is a cylinder with an axial bore in it giving diaphragm cap 110 an annular cross section. On its outer circumference is a step 112, which snaps into place against a matching step created on the wall of pump bore 102, thereby locking pump diaphragm cap 110 in place inside pump bore 102. The distal edge of cap 110 has a projection (knife edge) 114 that is forced into and compresses the material of pump diaphragm 108 holding the diaphragm firmly in place in groove 107′, thereby creating a tight seal that prevents the passage of air or liquid from one side of the pump diaphragm 108 to the other side. The hollow interior of pump diaphragm cap 110, the distal end of pump bore 102, and pump diaphragm 108 define the walls of pin cylinder 116 and pump chamber 118.

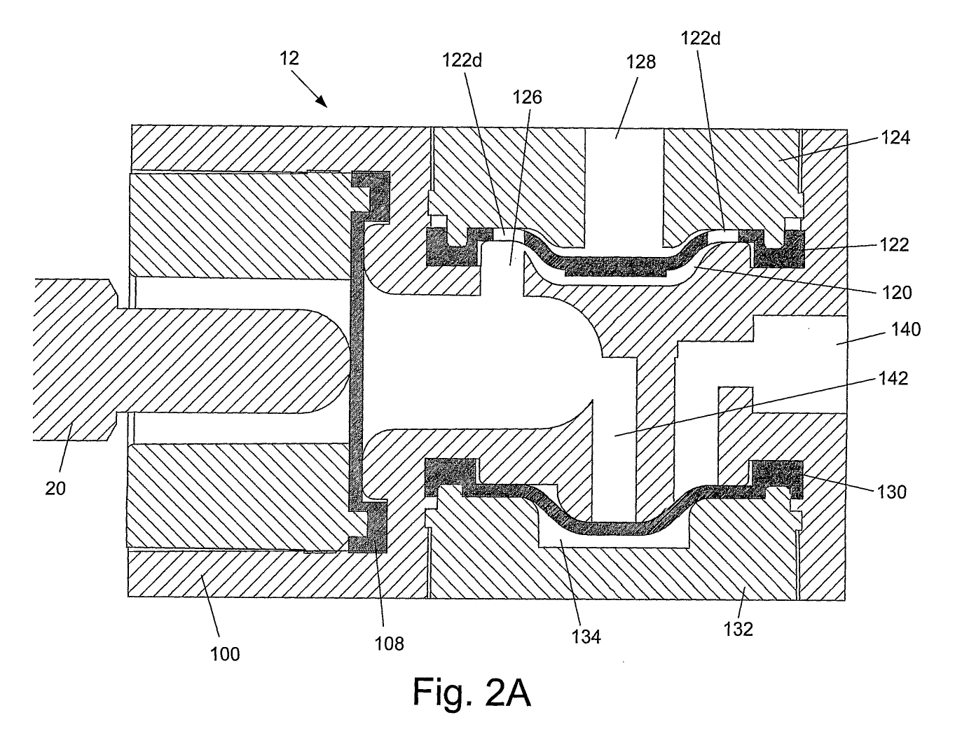

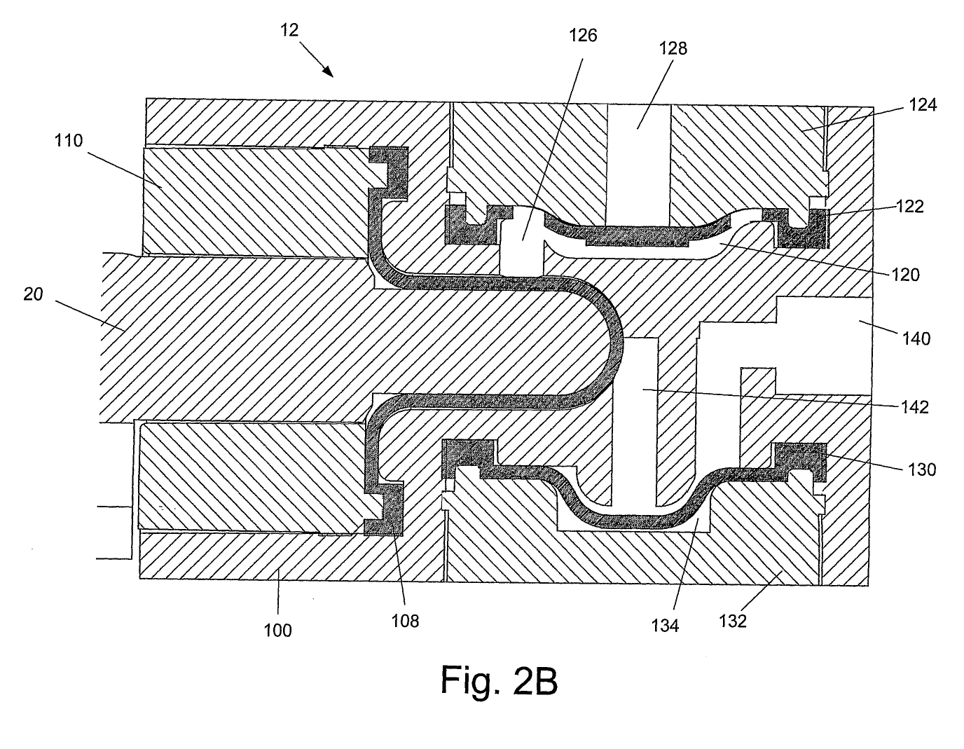

The inlet side of pump block 12 is similar in structure to the pump side. The inlet bore 104 has a distal end and a larger diameter proximal end. A seat is created at the interface for inlet diaphragm 122, which is held in place and sealed around its edges by inlet diaphragm cap 124. During the suction stage, inlet diaphragm 122 is pulled into inlet chamber 120 allowing liquid communication between pump chamber 118 and the liquid supply reservoir via channels 128 and 126. During the steady state (FIG. 1A) and the compression stages (FIG. 2B) inlet diaphragm 122 is pushed against the seat preventing liquid from flowing out of the reservoir through channel 128 into inlet chamber 120.

The structure of the outlet side of pump block 12 is similar to that of the inlet side, but in this embodiment there is the additional feature of a pressure sensor. At the bottom of outlet bore 106 is placed outlet diaphragm 130, which is held in place and sealed around its edges by outlet diaphragm cap 132, which has outlet chamber 134 created in its bottom side. On top of outlet diaphragm cap 132 is created a seat for pressure sensor diaphragm 136, which is held in place and sealed around its edges by pressure sensor diaphragm cap 138. During the compression stage (FIG. 1B and as will be described hereinbelow) the outlet diaphragm 130 moves into outlet chamber 134 allowing fluid communication between pump chamber 118, pressure sensor diaphragm 136, and the infusion set (patient) via channel 142, outlet chamber 134, and channels 141, and 140. During the steady state (FIG. 1A) and the suction stages outlet diaphragm 130 is pushed against the seat preventing liquid from flowing out of the pump chamber 118 through channel 142 into outlet chamber 134 and then through channels 141 and 142 to outlet channel 140.

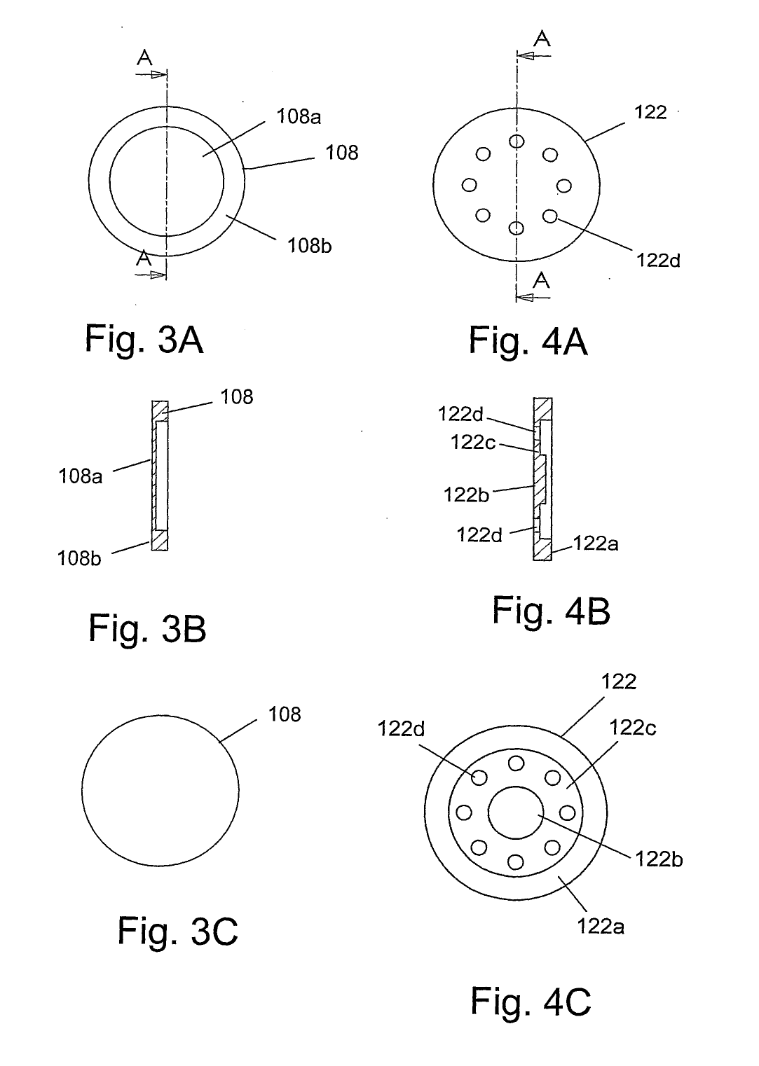

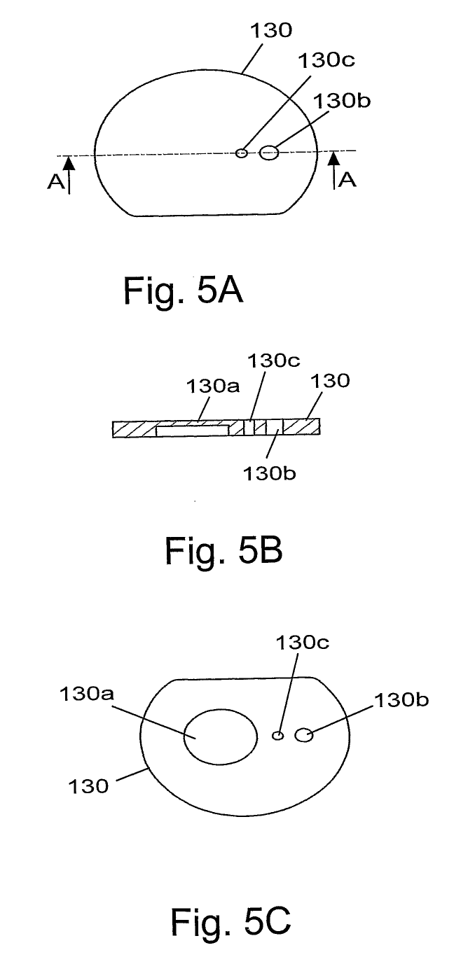

FIG. 2A and FIG. 2B are cross sectional views of a second embodiment of block 12 showing the suction and the compression stages respectively. This embodiment of the pump block is identical to that of FIG. 1A and FIG. 1B described hereinabove, with the exception that the pressure sensor is not a part of the pump block, but is located at another location in the insulin pump, preferably closer to the infusion set, as will be described hereinbelow. The absence of the pressure sensor in block 12 means that channel 141 is not needed and also that outlet diaphragm 130 (see FIG. 5A to FIG. 5C and description herein below) can be replaced with a diaphragm that is identical (with possibly a different diameter if necessary) to pump diaphragm 108 (see FIG. 3A to FIG. 3C and description herein below).

The operation of the pump can be explained by referring to FIG. 1A, FIG. 1B, FIG. 2A, and FIG. 2B. A pin on an eccentric gear, to be described hereinbelow, fits into slot 22 on pump pin 20. When the eccentric gear rotates, its rotational motion is turned into cyclic back and forth linear motion of pump pin 20 in pump pin cylinder 116. As pump pin 20 moves forward, its rounded distal end pushes against pump diaphragm 108 causing it to stretch and move forward, reducing the volume of pump chamber 118 and forcing liquid in the pump chamber into the outlet chamber 134 and towards the infusion set. As the eccentric gear continues turning, the pump pin 20 will be pushed to its extreme distal position (FIG. 1B and FIG. 2B) and then will be pulled back in the proximal direction. As the force exerted by the tip of pump pin 20 on pump diaphragm 108 is lessened, the internal forces created in pump diaphragm 108 by stretching it will act to return it to its minimum energy, i.e. steady state, position (FIG. 1A). As the pump diaphragm 108 moves in the proximal direction a vacuum is created in the gradually enlarging pump chamber 118 pulling inlet diaphragm 122 away from its seat allowing liquid to be drawn into the pump chamber from the reservoir through inlet chamber 120 (FIG. 2A).

FIG. 3A shows the internal face of the pump diaphragm 108, i.e. the face in contact with the liquid; FIG. 3B is a cross-sectional view along line A-A in FIG. 3A; and FIG. 3C shows the external face, i.e. the face in contact with the pump pin 20. Pump diaphragm 108 is made from a disc of elastomeric material, e.g. silicon. A section of the material on one side of the disc is removed to leave a thin central portion 108a, which can be repeatedly stretched up to five times its unstretched size and, when released, will return to its original size and shape and a thicker walled annular section 108b, which is pressed into grove 107′ by projection 114 on pump diaphragm cap 108. For typical diaphragm pumps of the prior art, the forward and backward movement of the diaphragm to produce the suction and compression stages is very small compared to the diameter of the diaphragm. The ratio of forward movement to the diameter of the pump diaphragm of the invention on the other hand, is very large. In order to emphasize this important point, in an embodiment of the invention, the pump diaphragm 108 moves back and forth 3 mm in a pump chamber 118 having a diameter of 2.4 mm.

In order to supply the force necessary to suck the liquid out of the reservoir and push it through the pump and infusion set into the patient, the pump pin 20 is made of a strong plastic material, e.g. reinforced nylon or polycarbonate, such that it is able to exert a force of 5 atmospheres and more when it pushes against the pump diaphragm 108. This creates a high pressure, which can open up occlusions in the infusion set or the cannula in the body. The dimensions and shape of the pin 20 and the pin cylinder 116 and pump chamber 118 are such that the pin fits tightly in the pin cylinder but can slide smoothly forward stretching the pump diaphragm 108 and causing an increase in its internal energy. As the pin 20 is pulled backwards, the energy stored in the diaphragm is released and it returns to its unstretched state. In the embodiment of the pump of the invention described above, the force exerted by the pump diaphragm 108 as it contracts is about two bars. Since only one bar is needed to create the vacuum in the suction stage, there is an excess of energy that is used to overcome friction. This is especially important in partially reusable embodiments of the pump of the invention described herein below in which the reservoir is a standard insulin cartridge. These cartridges contain rubber pistons which must be moved against considerable frictional forces as the liquid is drawn out of the cartridge. It is emphasized that, unlike standard piston pumps, the diaphragm is not attached to the pin and the only force required to be exerted by the motor is to move the pin back and forth and not to pull the diaphragm back, i.e. in the present invention the force required to create the suction is provided by the internal energy stored in the stretched diaphragm and not by the mechanism that moves the piston.

From the above can be appreciated one of the unique features of the present invention. The pump of the invention, although it superficially appears to comprise features of both conventional piston and diaphragm pumps used in prior art devices, is in fact neither of these. The significance of this is that it is possible to design the dosing pump of the invention using entirely different considerations of power requirements, strength of material, etc. than have been used to date in designing devices to accomplish the same task.

FIG. 4A shows the face of inlet diaphragm 122 that faces channel 128 that leads to the liquid reservoir; FIG. 4B shows a cross sectional view along line A-A in FIG. 4A; and FIG. 4C shows the face of inlet diaphragm 122 that faces inlet chamber 120. Inlet diaphragm 122 is a disc of elastomeric material, e.g. silicon. Material is removed from the disc to form three concentric zones as shown best in FIG. 4B. Zone 122a is a thick annular ring around the circumference of inlet diaphragm 122 that is pressed against the body 100 of pump block 12 by projections on the bottom of inlet cap 124 to hold inlet diaphragm 122 firmly in place. Zone 122c is an annular ring of relatively thin material that is very elastic and easily stretched by small forces. During the suction stage, a pressure differential is created on the two sides of inlet diaphragm which causes zone 122c to be pulled back away from its seat against the bottom of inlet cap 124. Zone 122b is a disc of intermediate thickness at the center of inlet diaphragm 122. During the suction stage zone 122b is also pulled back from its position against the end of channel 128, thereby allowing liquid to flow from the reservoir, through channel 128 and holes 122d in zone 122c, into inlet chamber 120 and from there through channel 126 into pump chamber 118. In the embodiment of inlet diaphragm 122 shown in the figures, there are eight holes 122d, but this number is not critical as long as there are sufficient holes in the diaphragm to allow enough liquid to be drawn into the interior of the pump block to fill the inlet chamber, pump chamber, and connecting channels during each suction step. At the end of the suction step, the pressure difference disappears and the internal forces in the stretched inlet diaphragm pull it back towards the seat on the bottom of the inlet cap 124. During the compression stage (FIG. 1B), zone 122b is pressed against the end of inlet channel 128 thereby preventing backflow of the liquid from the pump chamber into the reservoir. Zone 122b is thicker than zone 122c because it must be able to withstand without tearing the forces in the compression stage that are much greater than those of the suction stage.

The arrangement on the outlet side of block 12 works in a manner very similar to that of the inlet side. The major difference between the two sides being that the diameter (area) of the active sections of the outlet diaphragm 124 is much smaller than that of inlet diaphragm 122 since the pressure difference that is characteristic of the compression stage is much greater than that of the suction stage.

FIG. 5A shows the face of outlet diaphragm 130 that faces outlet chamber 134; FIG. 5B is a cross-sectional view along line A-A in FIG. 5A; and FIG. 5C shows the face of outlet diaphragm 130 that faces pump chamber 118. Outlet diaphragm 130 is a disc of elastomeric material, e.g. silicon. As best seen in FIG. 5B, material is removed from the disc to form a circular area 130a of relatively thin material that is very elastic and easily stretched by small forces. The thicker part of the diaphragm is pressed against the body 100 of pump block 12 by projections on the bottom of outlet cap 132 to hold outlet diaphragm 130 firmly in place. Circular area 130a is located at the bottom of the outlet chamber 134 that is created in the bottom of outlet diaphragm cap 132. During the suction stage, a pressure differential is created on the two sides of outlet diaphragm 130 which causes circular area 130a to be pushed against the end of conduit 142, thereby preventing liquid that enters the interior of block 12 from exiting through outlet chamber 134. During the compression stage (FIG. 1B), a pressure differential is created on the two sides of outlet diaphragm 130 which causes circular area 130a to be pulled back away from its seat against the bottom of outlet chamber 134, thereby unblocking the end of conduit 142. As pin 20 moves forward forcing the liquid out of the pump chamber, the liquid is pushed through channel 142 and pushes outlet diaphragm 130 away fro its seat at the bottom of outlet chamber 134. This allows the liquid to flow into outlet chamber 134 and from there; through channel 141 (via hole 130c in outlet diaphragm 130) the liquid is pushed through channel 140 (via hole 130b in outlet diaphragm 130) which leads to the infusion set.

Located below outlet diaphragm 130 (see FIG. 1A and FIG. 1B) is pressure sensor diaphragm 136. This diaphragm is made of similar material and has a similar shape to pump diaphragm 108. Its thinner section is however much thinner than that of the pump diaphragm in order to allow appropriate sensitivity changes in pressure. Pressure sensor diaphragm 136 is held in place against the top of outlet diaphragm cap 132 by pressure sensor diaphragm cap 138. One side of pressure sensor diaphragm 136 is open to channel 141. During the compression stroke liquid is forced into channel 141 causing pressure sensor diaphragm 136 to be pushed away from its seat and allowing the liquid to flow freely through outlet channel 140. In one embodiment the pressure sensor is an optical sensor. The pressure exerted on the diaphragm will cause it to move relative to an optical sensor (not shown in the figures), which measures the distance between the diaphragm and the sensor and translates the distance into pressure measurements. In other embodiments other types of pressure sensors are used, e.g. a strain gauge buried in the pressure sensor diaphragm can be used to measure its motion or a conductive silicone membrane, which changes electrical conductivity when stretched.

The output of the pressure sensor provides an indication that the insulin pump is operating properly and issues a real time warning, for example an audible signal, if a problem is detected. During the compression stage the liquid enters channel 141 and pushes pressure sensor diaphragm 136 closer to the pressure sensor, which detects the rise in pressure. The liquid then flows through channel 140 and passes into the body of the patient. and During the suction phase the diaphragm 136 will move back to its original position. This will be noted by the pressure sensor, which will signals that all is operating as it should. However, if there is a blockage (total or partial) in the infusion set, the liquid will not exit channel 140 at the predetermined rate and the pressure measured by the pressure sensor will not return to its basal level in a predetermined time period. On the other hand, if a leak develops in the infusion set, the pressure in the channel either will not rise or will rise and fall back to its basal level much faster than expected. Other problems, such as air in the system or lack of a constant supply of liquid from the reservoir will cause similar detectable phenomenon. In all of these cases, the abnormal rate at which the pressure difference on the two sides of pressure sensor membrane 136 rises and returns to normal will be measured and an alarm issued.

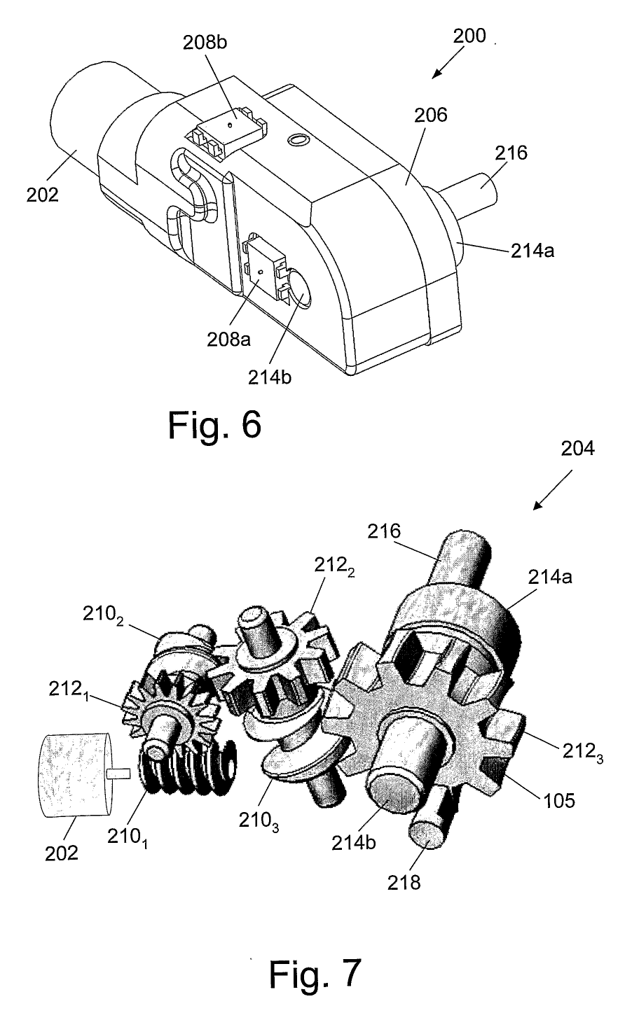

FIG. 6 shows the motor unit 200 of the dosing pump of the invention. Motor unit 200 comprises components that are responsible for providing the accurate motion, time of activation, and providing the power that is needed to create the pressure in the pump unit. The main features of motor unit 200 that are shown in the figure are: motor 202; housing 206, which surrounds a gear train; two sensors 208a and 208b, whose functions will be described hereinbelow; the front axle 214a and back axle 214b of the final gear in the gear train; and eccentric pin 216, which slips into slot 22 on pump pin 20 (see FIG. 1B).

The motor is a miniature D.C. motor. In an embodiment of the invention, the motor has dimensions of 6 mm diameter and 12 mm length. The motor is coreless, which means that it is very light, weighing only about two grams. The motor is very quiet and generates a moment of 0.1 milliNewton meter (mNm) and its shaft rotates independently the load at a speed of 28,000 rpm. In the present invention the voltage of 3V DC is supplied to the motor by the method of pulse width modulation (PWM) such that the moment is increased to 20 mNm. The motor speed is controlled by the duty cycle or pulse width. The moment is controlled by the input voltage (or current) and the number of revolutions of the shaft is very accurately controlled by the software and the controller, which stops the motor as required.

In FIG. 7 the housing 206 has been removed revealing the gear train 204 of the motor unit of the invention. All components of the gear train are built from hard plastic and weigh about two grams. The gear train has three stages, each of which comprises a worm gear 210_{1,2,3} and a spur gear 212_{1,2,3}. The first worm gear 210₁ is attached to the shaft of motor 202 and has a small diameter and small lead. The first spur gear 212₁ has small diameter and teeth, the second spur gear 212₂ has intermediate size diameter and teeth and the third spur gear 212₃ has large diameter and tooth size. The rear side of the axel 214b of the last spur gear 212₃ in the gear train has a relatively small diameter. However the side of the axle on the output side 214a is thick and strong in order to transfer enough moment to drive the pump pin by means of eccentric pin 216 located on output axle 214a. Eccentric pin 216 fits into slot 22 on pump pin 20 to cause the pump pin to move back and forth as eccentric pin 216 rotates.

In an embodiment, the gear ratio for each stage is 1:14, 1:10, 1:12.5 that is the overall gear ratio is 1:1750 Coupled with the motor described hereinabove, a moment of 8.0 mNM is produced at the output and the actual rate of revolution of the exit axle, after losses due to friction, is 20 rpm. When used with the pump unit described herein above, this speed of rotation is enough to supply 300 units of insulin in 10 minutes, or 1.0 unit of insulin is pumped for each revolution of output axle 214a.

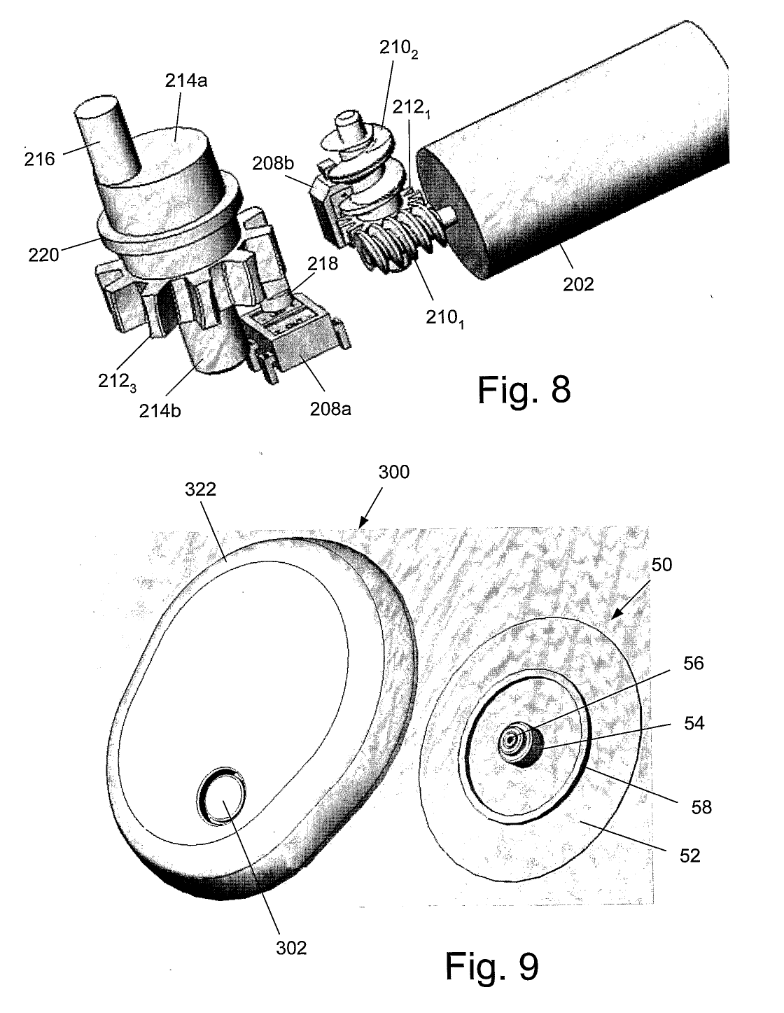

FIG. 8 illustrates the location and function of the sensors of the motor unit of the invention. In this figure only some of the gears are shown for clarity. On the back side of the last spur gear 212₃ is located pin 218. This pin is located exactly 180 degrees from eccentric pin 216 on the other side of gear 212₃. Sensor 208a detects each revolution of pin 218 in order to know exactly when eccentric pin 216 is in its maximum forward or backward position, i.e. when the pump unit changes over from the suction stage to the compression stage and vice versa. In the partially reusable embodiments to be described hereinbelow, the block of the pumping unit is discarded after one use and replaced while the motor unit is retained. The information from sensor 208a is needed to insure that the pump will stop with eccentric pin 216 in the exact position necessary for replacement of the block. Also shown in FIG. 8 is a seal 220 on axle 214a, which is needed to insure that the compartment containing the reusable components in the partially reusable embodiments of the invention is hermetically sealed. Sensor 208a and seal 220 are not needed in the disposable embodiments of the insulin pump.

Sensor 208b is located opposite the first set of gears and counts each tooth of spur gear 212₁ as it is rotated by motor 202. Because of the gear ratio that has been chosen, each time that sensor 208b counts one tooth on gear 212₁; this is equivalent to one revolution of the motor shaft. As said above, each revolution of the eccentric pin 216 is responsible for pumping 1.0 units of insulin from the reservoir into the patient. Since, for the gear train described herein it requires 1750 revolutions of the motor to produce one revolution of eccentric pin 216 and only half of the time is devoted to the compression stage, it follows that the combination of pump unit and motor unit described hereinabove pumps 0.0012 units of insulin per revolution of the motor. The signals from sensor 208b are sent to the internal control unit and used to determine how many revolutions the motor makes and to turn it off when the predetermined amount of liquid has been pumped. The presence of sensor 208b, which in effect counts revolutions of the motor, in combination with the input from the pressure sensor in the pump unit, which confirms that each compression stroke of the pump actually delivers the same, known volume of liquid, enable the dosing/insulin pump of the invention to deliver the liquid to the patient with an accuracy of 0.0012 units.

Sensors 208a and 208b can be any type of sensor known in the art. In the preferred embodiments of the invention sensors 208a and 208b are optical sensors. They can work by reflection, in which case each comprises a single element in which is located the source and detector, as in FIG. 8; or they comprise two elements a source on one side of the gear and a detector on the opposite side that detects when the light from the source is blocked by one of the teeth of the gear.

The internal control unit of the device of the invention is responsible for supplying insulin to the body of the patient according to predetermined programs. The internal control unit comprises a central processing unit (CPU) that has inputs from the sensors of the motor unit, pressure sensor of the pump unit and a switch that delivers a bolus dose. The CPU has outputs to the engine, sensors, and to means that provide an audible or other type of warning in case of malfunction of the device. The internal control unit comprises a 3 volt battery that supplies the energy necessary to perform all functions of the device, including activating the pump. As described hereinbelow, the invention is designed with either disposable or partially reusable embodiments. The energy requirements of the device are such that available disposable batteries will be able to provide all of the energy needed to operate the device for up to seven days. For partially reusable embodiments of the device, rechargeable batteries that can be recharged by any method known in the art are provided. In some embodiments of the invention the patient will be able to change the dosage during use according to the nature of his activity at a particular time of day by selecting one of five basal doses from a remote control unit.

Components are provided that allow two way communication with the device. These components are used, amongst other things, to update or change the bolus dose and basal programs and to download information, such as when doses were administered and the quantity of insulin in each dose and how many and when boluses were given from the memory of the CPU. The communication components can be any means known in the art, e.g. a USB connection or based on RFID (preferred for disposable devices because of its relatively low cost) or Bluetooth technologies. Depending on the embodiment, the input/output to the device can be managed entirely or partially using input means and a display screen that are part of the partially reusable device, a dedicated remote control unit, or a PC or laptop computer equipped with appropriate software. In preferred embodiments of the invention, the remote control unit is a multipurpose handheld device such as a Palm PC or a mobile phone using a standard operating system such as Windows, Unix, or Linux to which the dedicated software needed to manage the input/output to the device is downloaded from a disk-on-key (or other standard means) supplied with the device of the invention.

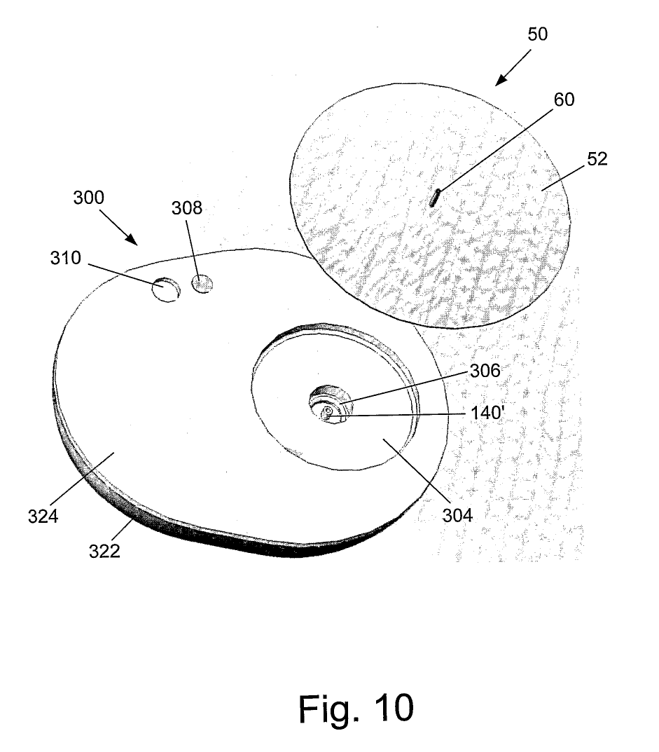

FIG. 9 and FIG. 10 are general views showing a disposable embodiment of the insulin pump 300 of the invention and an infusion patch 50 from the top and bottom respectively. Disposable pump 300 comprises the embodiment of the pump block that does not have the integral pressure sensor. All input/output to the device is from a remote control unit, preferably using RFID or Bluetooth connection to a standard Palm-like device or mobile phone. The one exception to this is that a bolus dose can be administered manually by pressing on button 302 that can be seen on the cover of the pump. The bolus button can be used to administer pre-set volumes of insulin for each pressing of the button if the remote control unit is temporarily unavailable.

Pump 300 comprises the components described hereinabove packaged in an oval shaped plastic case made up of cover 322 hermetically sealed to base 324. Pump 300 because of its small size and weight can be attached directly to infusion patch 50 and does not have to be supported at some other location on the body and connected to the infusion patch by tubing.

Infusion patch 50 is a standard patch, e.g. an ICU Orbit 90 insulin patch. It comprises a circular adhesive pad 52 with an adhesive on one side for attaching it to the body of the patient. On the opposite side of adhesive pad 52 is a circular plastic base 58. Post 54 is located on the base 58. A channel passes through post 58 to provide liquid communication between port 56 at the top of post 58 and cannula 60 on the adhesive side of pad 52. The outlet chamber 140 of pump 300 is connected via port 56 to cannula 60, which is introduced subcutaneously into the patient.

Referring to FIG. 10, two coaxial sockets have been created in the bottom of pump 300. Shallow, larger diameter socket 304 fits over base 58 of infusion patch 50 and deeper smaller diameter socket 306 fits over post 54. In the center of socket 306 is located the distal end of exit pipe 140′, which at its proximal end is connected to the outlet channel 140 of pump bock 12. Also seen on the bottom of pump 300 are the end of a silicon plug 308, which is used to fill the reservoir of pump 300 with insulin and a region 310 of the base 324 of the pump that is made thin as a safety precaution. The use of these features will be explained hereinbelow.

When pump 300 is fitted over the post and base of the infusion patch 50, the end of exit pipe 140′ fits tightly into port 56 completing the path from the pump block 12 into the body of the patient. In embodiments of the invention, a quick release mechanism (not shown in the figures) is provided to allow the pump 300 to be easily disconnected from and reconnected to the infusion patch 50 for activities such as swimming, sport, etc.

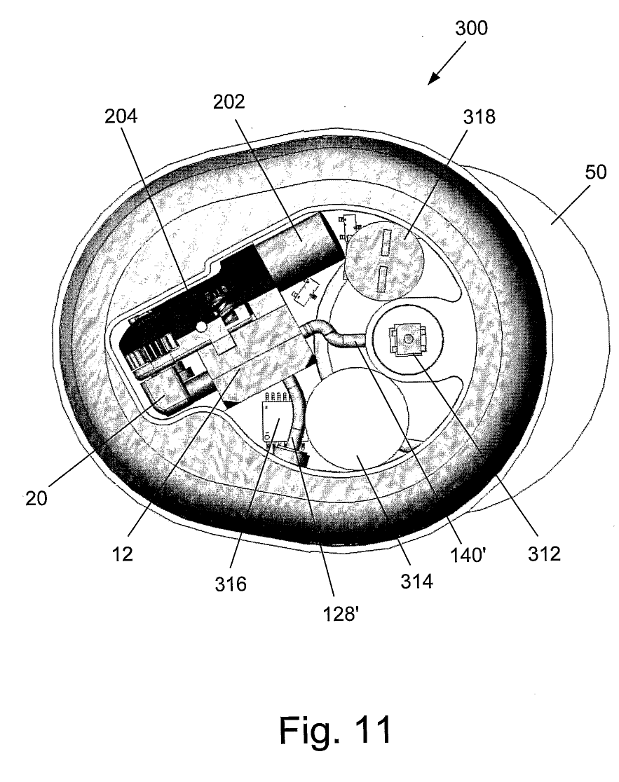

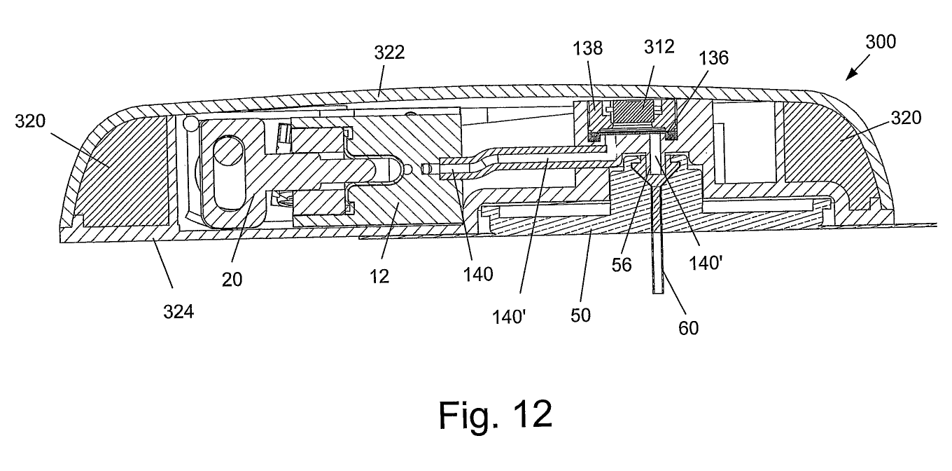

FIG. 11 is a top view of a disposable embodiment of the dosing/insulin pump of the invention with part of the cover removed to expose some of the internal components and FIG. 12 is a cross-sectional view of the same pump attached to an infusion patch.

In FIG. 11 can be seen pump block 12, pump pin 20, gear system 204, and motor 202. Also seen are pressure sensor 312 and the principal electronic components: CPU 316, which includes the communication means to the remote control unit; battery 314; and buzzer 318 which signals if a malfunction has occurred. Also shown is tube 140′, which is connected to the outlet channel of pump block 12 and tube 128′, which connects the infusion reservoir to the inlet channel of pump block 12. Tubes 128′ and 140′ can be made of any suitable material known to skilled persons, e.g. silicon tubing.

In this embodiment of the disposable infusion pump of the invention, the reservoir is a doughnut-shaped collapsible elastomeric, e.g. silicon, bladder 320 (FIG. 12) that is placed around the perimeter of the pump against the inside of the cover surrounding the other components. When full, bladder 320 holds 5 ml of insulin, which is normally enough for three days use, after which the pump 300 and infusion patch 50 are removed from the patient, discarded, and replaced with a new patch and pump in accordance with FDA regulations. From FIG. 12 it can be seen how the sensor 312 is located within pressure sensor diaphragm cap 138 on top of pressure sensor diaphragm 136. During the compression stage, insulin is forced out of pump block 12 and enters tube 140′. The insulin flows through tube 140′, lifts pressure sensor diaphragm 136 changing its distance to pressure sensor 138 as explained hereinabove, and continues flowing through port 56 of infusion patch 50 and through cannula 60 into the body of the patient.

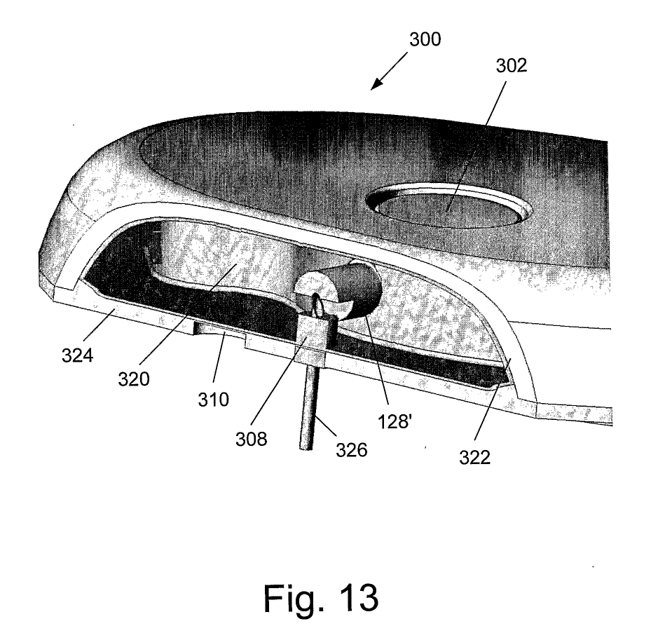

FIG. 13 is a view of disposable pump 300 of the invention with part of the side sliced off to reveal the inside of bladder 320 and illustrates the method of filling the bladder with insulin. The disposable pump 300 of the invention is supplied with the inflatable silicon bladder 320 evacuated and collapsed. Before attaching pump 300 to infusion patch 50, the patient draws insulin out of a standard vial with a syringe, pushes the pointed end of syringe needle 326 through silicon plug 308 into the interior of the bladder, and pushes on the piston of the syringe filling bladder 320 with insulin. Note that the hard plastic end of tube 128′ projects into the interior of the bladder over plug 308 to prevent the tip of needle 326 from puncturing the bladder. The pump is self primed by pushing on the piston of the syringe until all the air is pushed out and the interior of the pump unit and all channels and tubes for conducting liquid through the pump are filled with insulin as evidenced by one or more drops of insulin exiting from the end of tube 140′. The needle 326 is then pulled back out through silicon plug 308, which is self-sealing, preventing air from entering or insulin from escaping the interior of bladder 320.

Because the insulin reservoir is a collapsible silicon bladder, theoretically if the pump 300 receives a sharp blow or is compressed in some manner, the pressure in the bladder could exceed five atmospheres, in which case insulin could be forced through the pump block and into the patient in an uncontrolled and unwanted manner. Therefore, as a safety measure to avoid such a potentially dangerous occurrence, a portion 310 of the base 324 of the case of pump 300 is made thin, such that it will not support the wall of the bladder, which will attempt to expand against the interior of the pump. If the internal pressure in the bladder exceeds a predetermined value, then the lack of support at 310 will allow bladder 320 to burst at that location.

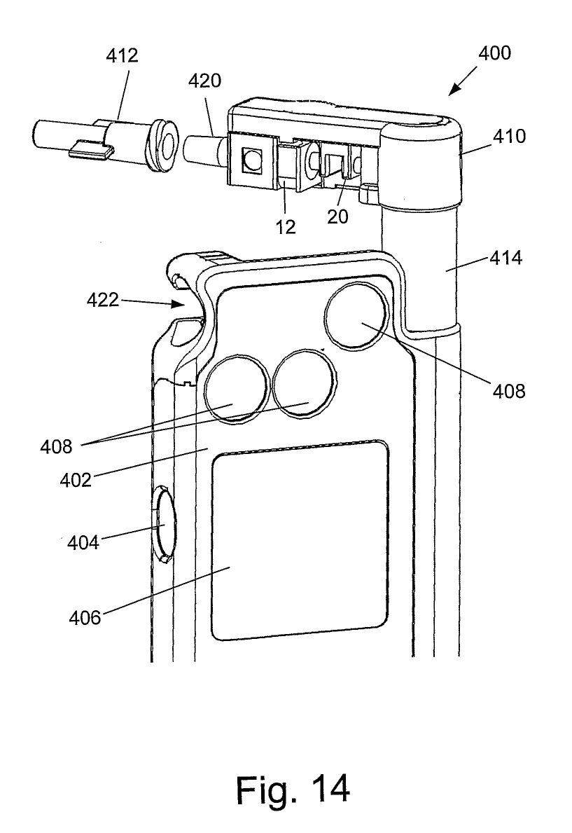

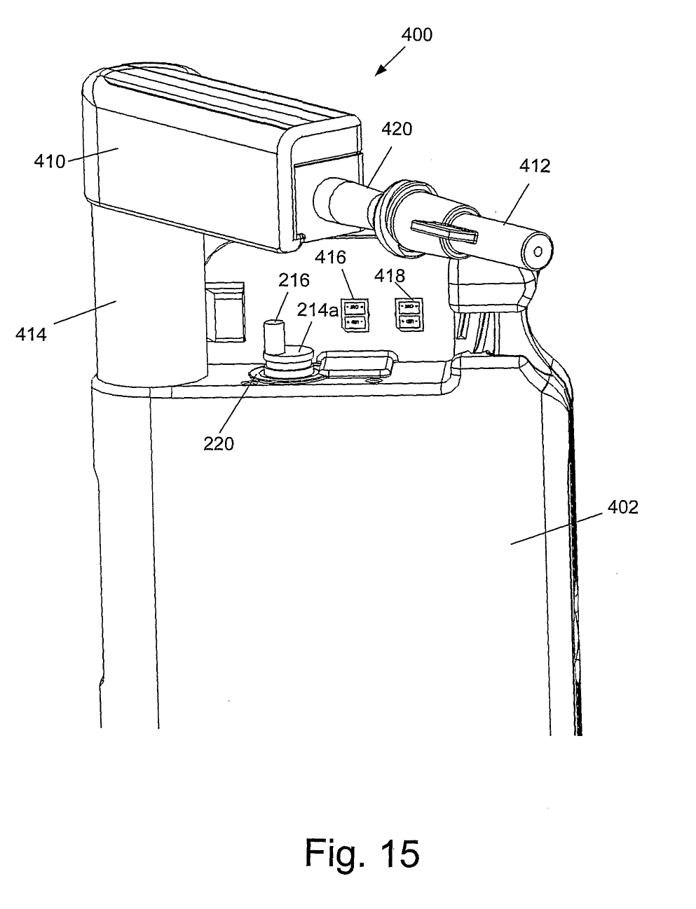

FIG. 14 and FIG. 15 show an embodiment of a partially reusable dosing/insulin pump 400 of the present invention. Pump 400 is comprised of reusable section 402 and disposable section 410.

Reusable section 402 comprises a sealed compartment in which are located the internal control unit, motor, and gear system. On the outside of the case of reusable section 402 can be seen in the figures bolus button 404, display screen 406, and various control buttons 408, e.g. on-off, basal program, increase or decrease value, program menu select or activate. Not shown are a USB port for connection to an external computer to reprogram the internal CPU and download historical data and a port for connecting to an external electricity source to recharge the batteries. Sticking up through the top of reusable section 402 is the axle 214a of the final gear in the gear stem with the attached pin 216. Seal 220 fits around axle 214a to prevent water or other fluids from entering the sealed compartment.

The reservoir for pump 400 is a standard 3 ml insulin pen cartridge 414. Cartridge 414 fits into a cylindrically shaped bore, which is located outside of the sealed compartment on the side of reusable section 402.

Disposable section 410 of pump 400 comprises pump pin 20, pump block 12, and the pressure sensor diaphragm. At one end of disposable section 410 is a cylindrical bore that is designed to fit over the end of cartridge 414. At the center of the bottom of this bore is a port that is connected by tubing to the inlet channel 128 of the pump block 12. At the other end of disposable section 410 is a nipple 420 in fluid communication on the proximal side to the outlet channel 140 of pump block 12 and on the other side with a connector 412 connected to the tubing (not shown) of the infusion set.

The pump 400 is assembled as follows: First cartridge 414 is inserted—piston end first—into the bore in reusable section 402. Then disposable section 410 is slid over the other end of cartridge 414. Now the infusion set is connected to nipple 420, without the distal end of the infusion set connected to the cannula into the body of the patient. Now the top of disposable section 410 is pushed downwards causing the end of cartridge 414 to slip into the port designed to accommodate it thereby completing a fluid path from the inside of cartridge 414 to inlet channel 128 of pump block 12. At the same time disposable section 410 is rotated inwards towards the upper part of reusable section 402 until eccentric pin 216 slips into the slot in pump pin 20 and connector 412 snaps into and is locked in opening 422 of reusable section 402. Sensor 416 detects if the disposable section 410 and the reusable section 402 are properly locked together. When this occurs, the pressure sensor diaphragm is opposite pressure sensor 418, which will indicate if there are any problems with path for the insulin as described hereinabove.

At the bottom of the bore in reusable section 402 is a projection that fits into the interior of cartridge 414 pushing against the piston. The lengths of the bores in disposable section 410, in reusable section 402, and of the projection are such that, when the pump 400 is assembled, the piston in cartridge 414 is pushed just enough to force a predetermined amount of insulin out of cartridge 414, through pump block 12 and the tubing of the infusion set, thereby self-priming the system. It is to be noted that after the self-priming step takes place, the piston is no longer pushed forward to expel insulin from the cartridge, rather the piston is pulled forward by the vacuum created as the insulin is sucked out of the cartridge during the suction stage of the operation of pump block 12.

Partially reusable pump 400, although much lighter and smaller than presently commercially available insulin pumps, is nonetheless larger and heavier than the disposable pump 300 of the invention. Therefore it must be worn attached to the patient's belt, hanging from a necklace, or by some equivalent means and connected by tubing to the infusion patch. After the insulin in cartridge 414 is used up, the above described procedure is carried out in reverse to separate the disposable and reusable parts of pump 300. Disposable section 410, pen cartridge 414, and the infusion set are disposed of and new ones are attached to the reusable section 402.

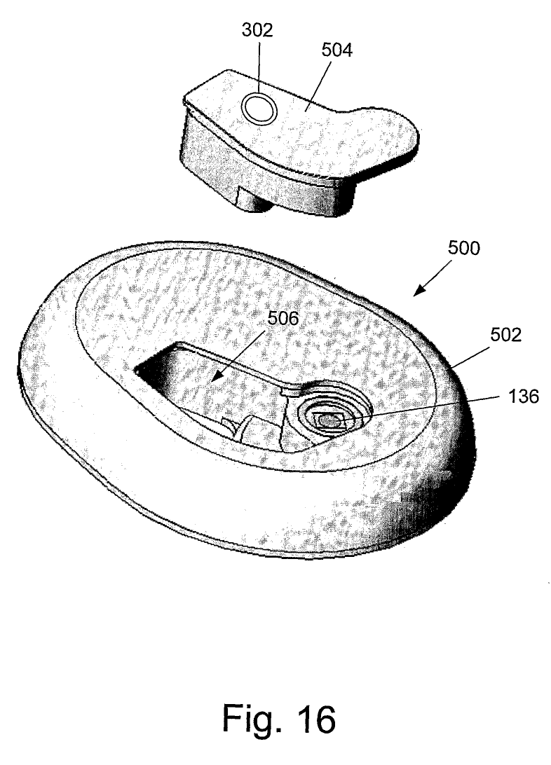

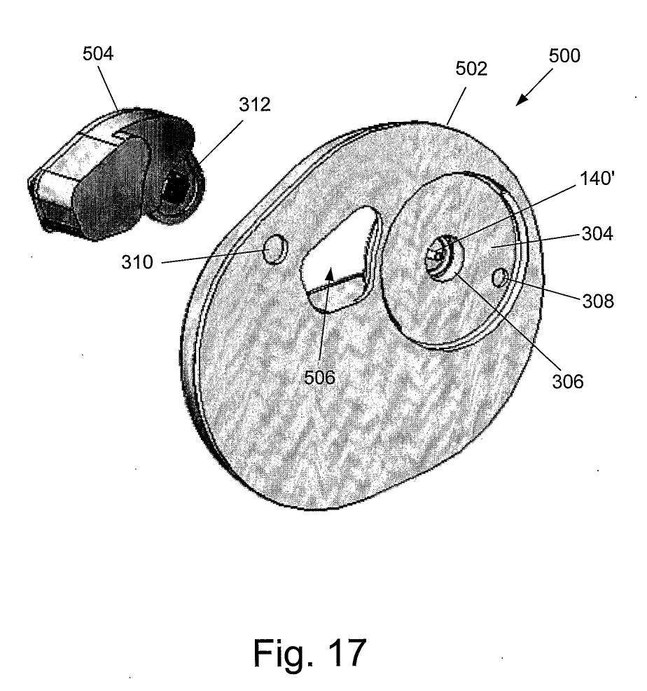

FIG. 16 and FIG. 17 show top and bottom views respectively of another embodiment of a partially reusable pump 500. This embodiment is very similar to the completely disposable insulin pump 300 described hereinabove and shown in FIG. 9 to FIG. 13. In this embodiment pump 500 is comprised of two sections: disposable section 502, which snaps directly onto an infusion patch in the same manner as pump 300 as shown in FIG. 10 and reusable section 504. Reusable section 504 comprises the. bolus button 302, the CPU, optical pressure sensor 312, a transmitter/receiver for two-way communication to the remote unit, and a buzzer. The disposable section comprises the pump block, gear unit, motor, pressure sensor diaphragm 136, reservoir, and battery.

As in fully disposable pump 300, the reservoir is a doughnut shaped bladder that is filled using a syringe via plug 308. Also provided in the case surrounding disposable section 502 is an area of reduced wall thickness 310 as a safety feature.

Both sections 502 and 504 are hermetically sealed and section 504 fits tightly into socket 506 in the disposable section 502. Matching electrical contacts (not shown in the figures) on the outside of reusable section 504 and the walls of socket 506 are brought in contact with each other as section 504 is inserted in socket 506, thereby completing the electrical circuits.

In the preferred embodiment of partially reusable dosing/insulin pump 500, the remote unit is a hand held device such as a palm-pilot or a mobile telephone and the two-way communication between the remote unit and reusable section 504 is based on Bluetooth technology.

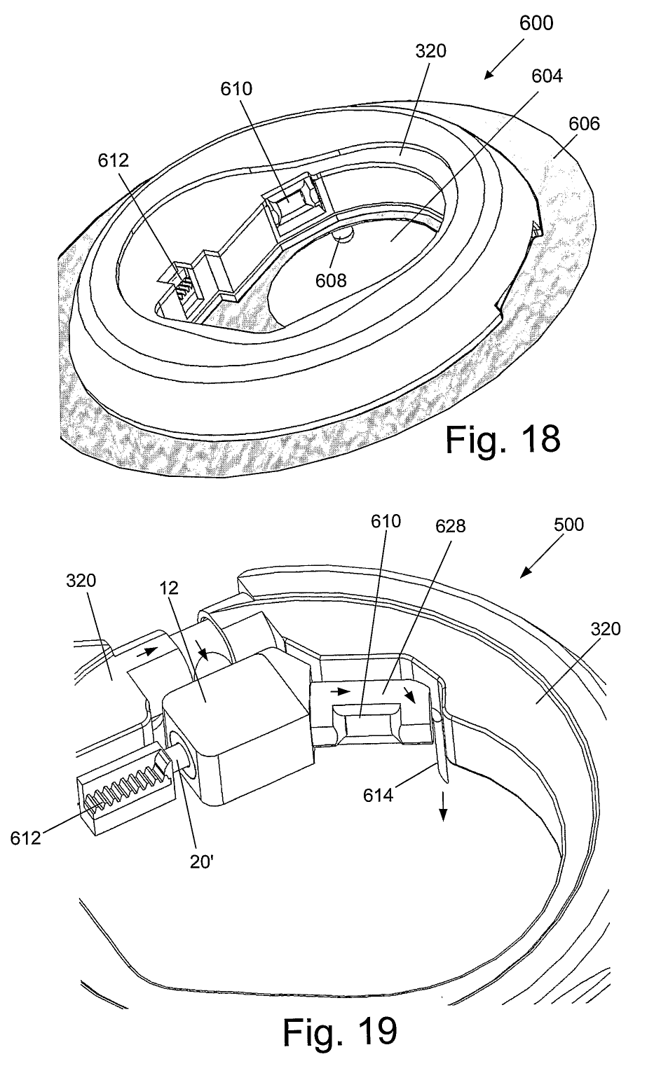

FIG. 18 to FIG. 26 show another embodiment of a partially reusable dosing/insulin pump according to the present invention. Most of the components of this embodiment are the same as or very similar to those that have been described herein above for other embodiments of the invention; however there are some significant differences that will be described herein below. The most significant of these are changes in the gear system that allow the reciprocating motion of the pump pin to be caused by a rack and pinion gear instead of by the rotary motion of an eccentric pin as described herein above. Additionally, there are other changes that have allowed reducing the overall dimensions especially the height, of the device.

In this embodiment, as opposed to the embodiment shown in FIG. 9 to FIG. 13 and the embodiment shown in FIG. 16 and FIG. 17 the annular disposable part 600 has an adhesive pad 606 directly attached, e.g. glued to its bottom surface and a small hollow needle 614 in liquid communication with the output channel of the pump block projects downward from it for penetrating the skin, thereby eliminating the need for a separate infusion set and an arrangement for connecting to it.

FIG. 18 and FIG. 19 show the disposable part 600 of a partially reusable dosing/insulin pump with part of the cover removed to show the main components. Shown in these figures are the block 12′ of the pump unit, the rack gear 612, the pump pin 20′, a silicon connection piece 628 with a pressure sensor diaphragm 610 that is created in its side, the adhesive pad 606, needle 614, and an a doughnut shaped elastomeric, e.g. collapsible silicon bladder 320, which is positioned around the outer perimeter of disposable part 600. The center of disposable part 600 is hollow defining a socket 604 into which the reusable part 602 can be inserted. The small arrows in FIG. 19 show the direction of the flow of insulin from the bladder, through the pump block and needle, into the patient. All of the components of disposable part 600, except adhesive pad 606 and the part of needle 614 that extends below the adhesive pad, are enclosed within a waterproof plastic case.

The disposable part 600 is supplied in a sterile wrapping with bladder 320 empty, needle 614 covered by a protective cover 608 (FIG. 18), and the bottom adhesive surface of pad 606 covered with a protective peelable layer. Prior to use disposable part 600 is removed from its wrapping, bladder 320 filled with a predetermined, e.g. 3 cc or 5 cc of insulin as described herein above (see FIG. 13). reusable part 602 is inserted into socket 604, the peelable protective layer is removed from adhesive pad 606, protective cover 608 is removed from needle 614, the insulin pump is pressed against and affixed to the skin of the patient, and the pump is activated.

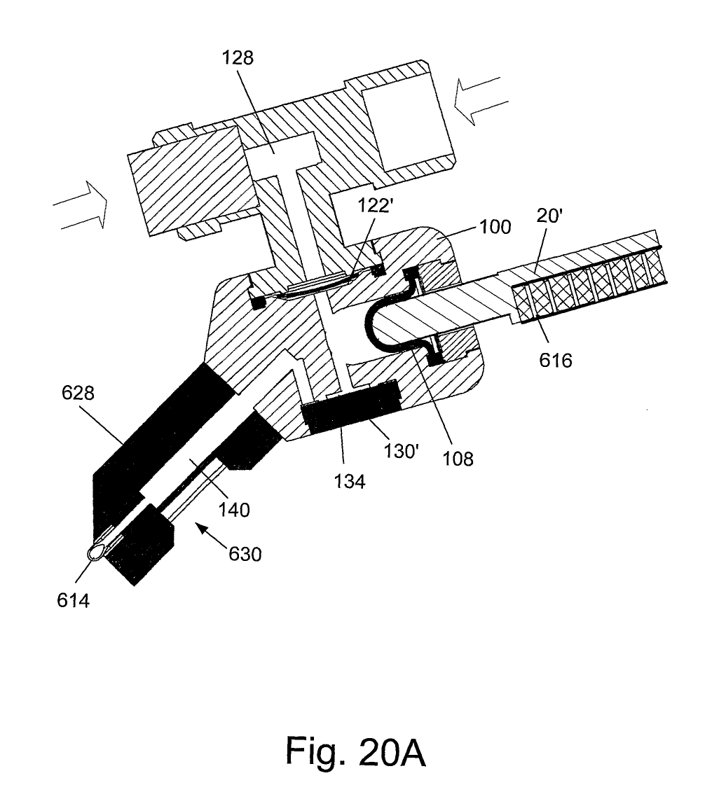

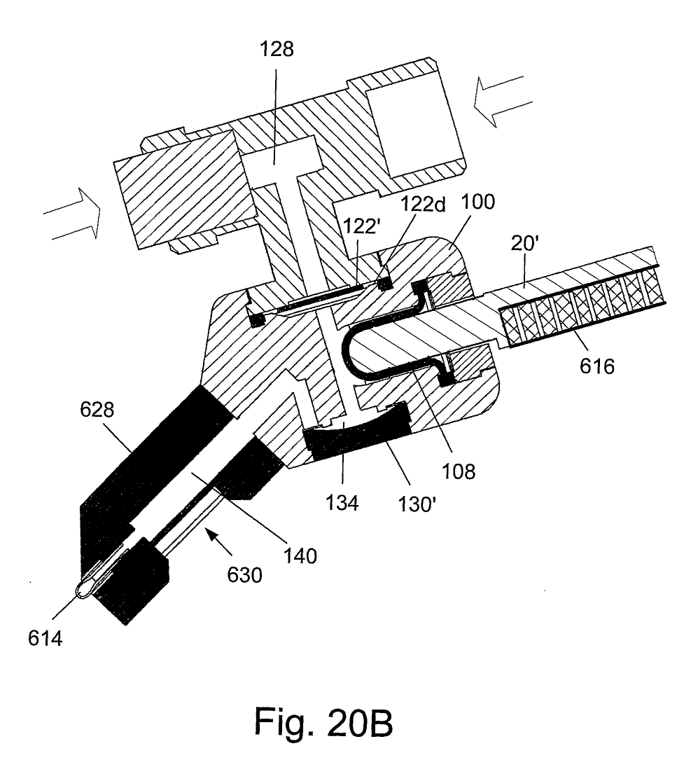

FIG. 20A and FIG. 20B are cross sectional views of the pump unit of the device of FIGS. 18 and 19 showing the suction and the compression stages respectively. The pump unit functions essentially the same as described herein above with reference to FIGS. 1A, 1B, 2A, and 2B. The principal features shown in FIG. 20a and FIG. 20B are the pump block 100, pump diaphragm 108, input diaphragm 122′, output diaphragm 130′ pump pin 20′ and the pinion gear 616. The double arrows indicate the connections of the bladder 320 to pump block 100. The silicon connection piece 628 has a hollow channel created through which is part of conduit 140 from outlet chamber 134 to the needle 614 whose upper part is firmly embedded in connection piece 628. The side wall of conduit 140 through connection piece 628 at approximately the location indicated by arrow 630 in FIG. 20A and FIG. 20B is made very thin so that it will move in and out as the pressure changes in conduit 140. This thin wall section is the pressure sensor diaphragm 610 in FIGS. 18 and 19. Rack gear 616 is an integral part of pump pin 20′. They are manufactured as a single piece out of plastic.

In embodiments of disposable unit 600 the components of the pump block are made of medical polypropylene, the diaphragms of silicon, and the needle is made of 31 gauge stainless steel.

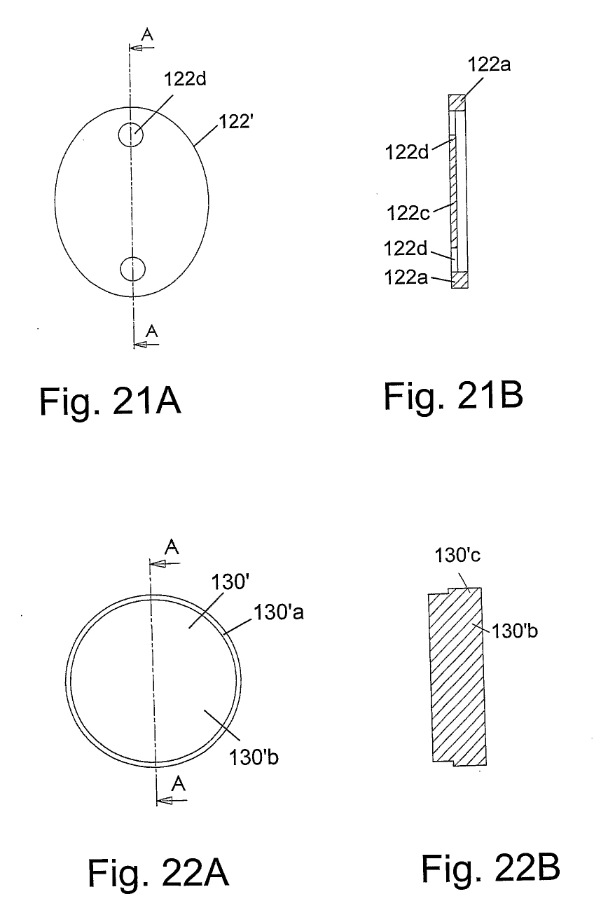

FIG. 21A and FIG. 21B show different views of the inlet diaphragm of the pump unit of FIGS. 20A and 20B and FIG. 22A and FIG. 22B show different views of the outlet diaphragm of the pump unit of FIGS. 20A and 20B. Comparing these figures with FIGS. 4A to 5C that show the comparable diaphragms it can be seen that those in the presently described embodiment are much simpler. In particular, inlet diaphragm 122′ has only two holes 122d to allow liquid to flow from the reservoir into the inlet chamber during the suction stage (FIG. 20A). Making the shape of diaphragm 122′ elliptical instead of circular allows the distance between holes 122d to be increased while also reducing the overall volume of the inlet side of the pump unit. Outlet diaphragm 130′ has no holes. It is made of soft silicon, which allows it to be pushed into the outlet chamber 134 allowing liquid to flow from the pump chamber to the needle 614 as show in FIG. 20B.

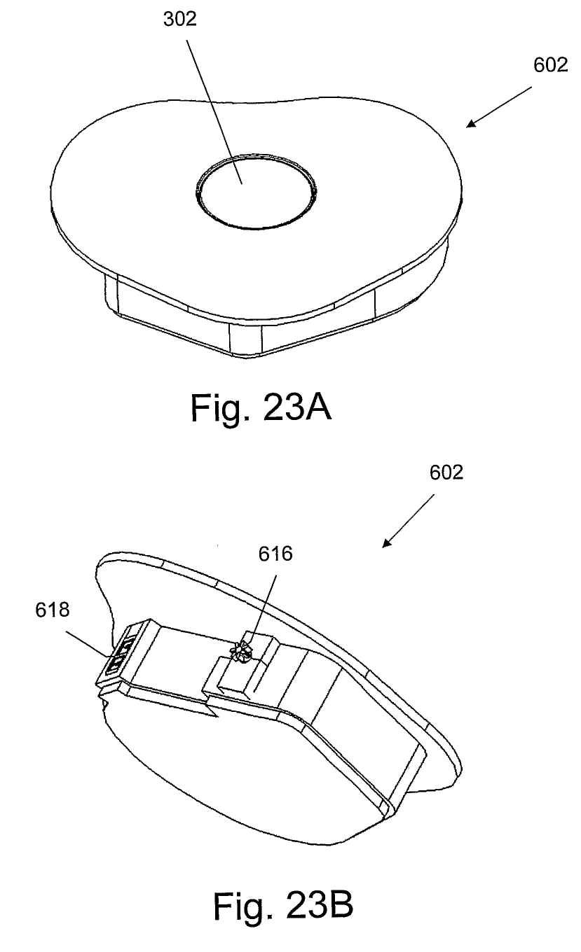

FIG. 23A and FIG. 23B respectively show the top and bottom of the reusable part 602 of the partially reusable dosing/insulin pump. All components of reusable part 602 are enclosed in a waterproof plastic case. On the top surface of the case is a bolus button for manually injecting a predetermined volume of liquid. Pressing on the bolus button for an extended period of time, e.g. ten seconds, activates (or deactivates) the CPU inside the reusable part, which turns on (or off) the pump according to a preprogrammed schedule. On the sides of reusable part 602 are the pressure sensor 618, which will be opposite pressure sensor diaphragm 610, and pinion gear 616, which will engage rack 612, when reusable part 602 is inserted into socket 604 in disposable part 600.

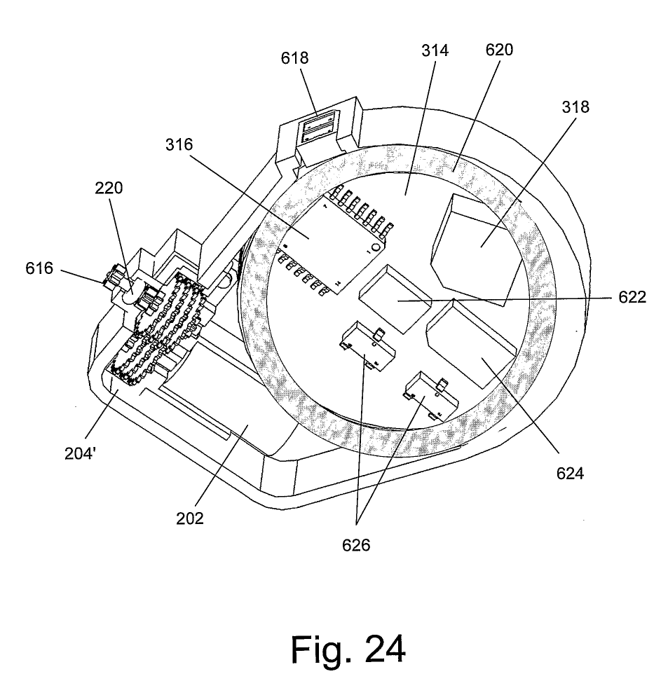

FIG. 24 shows the internal components of the reusable part 602 of this embodiment of a partially reusable dosing/insulin pump. Seen are a D.C. reversible electric motor 202 that drives the gear assembly 204′, pressure sensor 618, and electric components mounted on top of a rechargeable lithium battery 314. The wiring circuit that interconnects the various electrical components of reusable part 602 is not shown. The electrical components include charging coil 620 and charge control chip 622 for recharging the battery, alarm 318 to signal a malfunction of the pump, a central processing unit 316 that controls the operation of the pump, a Bluetooth communication chip 624, and transistors 626. The CPU can be reprogrammed from a remote control station, e.g. a PDA or cellular phone, as described for other embodiments of the invention herein above. The pressure sensor 618 can be of any type known in the art, e.g. an optical sensor comprised of a LED and detector to detector the light waves reflected from the pressure sensor diaphragm 610. Similarly an ultrasonic transducer can be used to emit and detect reflected ultrasound waves. An “O” ring seal 220 is located on the shaft of the gear system 204′ before pinion gear 616 to prevent water from entering the interior of reusable part 602 if the patient wants to take a bath or shower or go swimming while the dosing/insulin pump is attached to his body and operating.

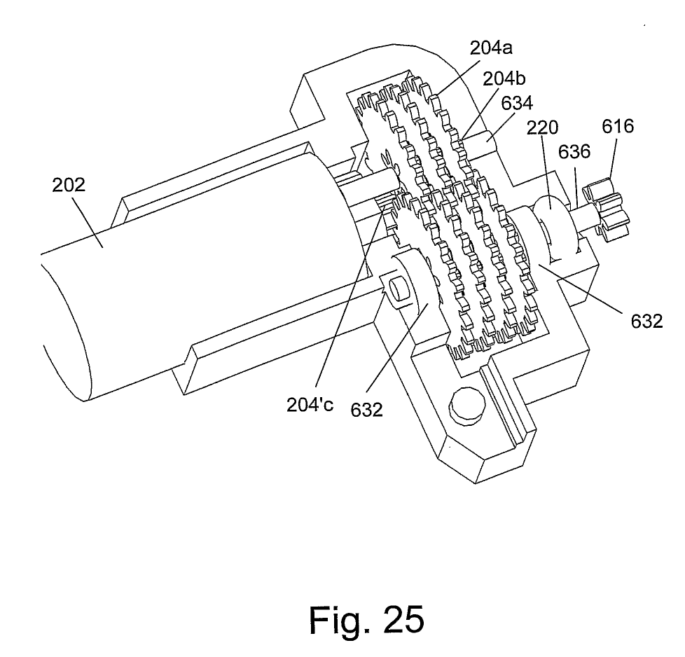

FIG. 25 shows an enlarged view of the motor 202 and gear train 204′ of FIG. 24. The gear train comprises seven large straight cut spur gears 204′a each having 24 teeth. Each gear 204′a has a smaller straight cut spur gears 204′b each having 8 teeth fixedly attached to it. Three of the pairs of gears 204′a and 204′b turn freely on axle 634 which does not rotate and is fixedly attached to the plastic walls of the reusable part 602. The other four pairs of gears are mounted on axle 636 which is supported by bushings 632. The first three pairs of gears on axle 636 rotate freely, the fourth (last) pair is fixedly attached to axle 634 causing the axle and the pinion gear 616 to rotate. An additional small gear 204′c is fixedly attached to the shaft of motor 202. The teeth of gear 204′c engage those of the first gear 204′a on axle 636. The total gear ratio is 1:5500, i.e. 5,50 revolutions of the shaft of motor 202 causes one complete revolution of the spur gear 616 at the end of the gear train 204′. One complete turn of spur gear 616 causes the rack gear 612 to move all the way forward, pushing the piston pin 20′ to the extreme forward position and, in this embodiment causing 1.5 units of insulin to be injected into the patient. Reversing the motor 202 causes the rack and pin to move backwards drawing 1.5 units of insulin out of bladder 320. A mechanical counter (not shown in the figures) is attached to gear 204′c on the shaft of the motor in order to count the number of revolutions of the motor and transfer this information to the CPU that uses it to turn off the pump when the desired amount of insulin has been injected. The CPU in the reusable part of this embodiment utilizes the stall current, i.e. the rise in current drawn by the D.C. motor when the spur gear reaches the end of the pinion gear, to reverse the direction of the motor. In this embodiment, the gears 204′a, 204′b, 204′c and 616 and axles 634 and 636 are made of metal, e.g. stainless steel. Bushings 632 are of another type of metal, e.g. bronze. In other embodiments some or all of these components can be made of other types of material, e.g. plastics or ceramics.

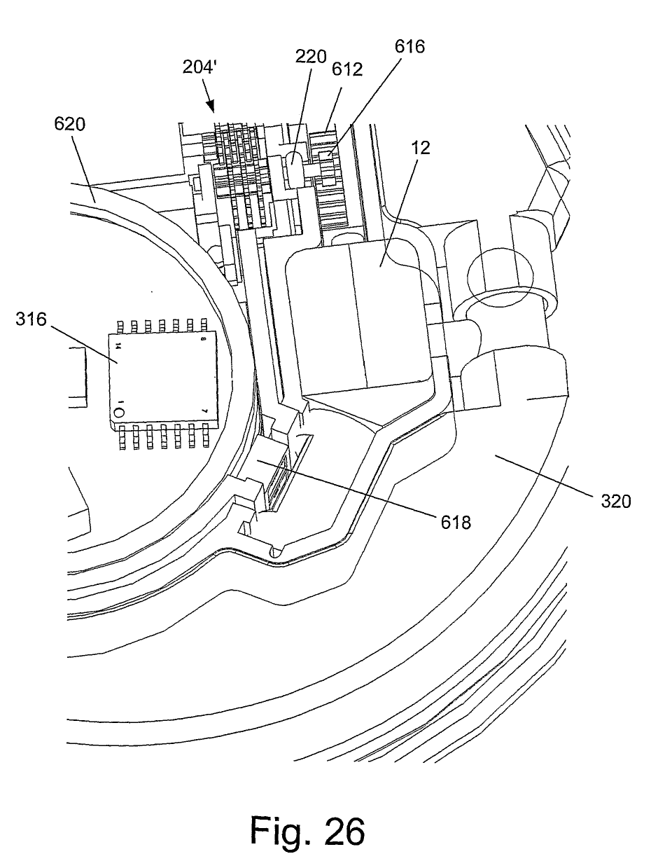

FIG. 26 shows the reusable part 602 of this embodiment of a partially reusable dosing/insulin pump inserted into the socket 604 in the disposable part 600. The covers of both parts have been removed to show how the teeth of the pinion gear 616 mesh with the teeth on the rack 612 and the pressure sensor 618 lines up opposite the pressure sensor diaphragm (not visible in FIG. 26).

Although embodiments of the invention have been described by way of illustration, it will be understood that the invention may be carried out with many variations, modifications, and adaptations, without exceeding the scope of the claims.

1. A dosing pump that can be worn on the body of a patient for subcutaneously delivering liquid with centi-micro liter accuracy to the body of said patient, said dosing pump comprising:
A. a pump unit comprising:
i) a pump block comprising:
a.) a pump chamber with a pump diaphragm stretched across its entrance;
b.) a pump pin cylinder extending from the exterior of said pump block to said entrance to said pump chamber;
c.) an inlet chamber with an inlet diaphragm stretched across its entrance;
d.) an outlet chamber with an outlet diaphragm stretched across its entrance; and
e.) several channels to provide fluid communication between said pump, inlet, and outlet chambers and the outside of said pump block;
ii) a pump pin comprising a rounded distal end that is located in said pump pin cylinder;
iii) a motor unit comprising:
a.) a motor; and
b.) a gear system comprising a plurality of gears arranged to transfer the rotary motion of the shaft of said motor to an output axle coupled to said pump pin;
B. an internal control unit; and
C. a reservoir containing said liquid;
wherein, when said motor is activated, the rotational motion of said motor and the gears in said gear system is transformed into cyclic back and forth linear motion of said pump pin in said pin cylinder; wherein, as said pump pin is pushed forward, its rounded distal end pushes against said pump diaphragm causing said pump diaphragm to stretch and move forward, reducing the volume of said pump chamber, increasing the pressure on liquid located in said pump chamber causing said outlet diaphragm to stretch into said outlet chamber, thereby allowing said liquid to flow out of said pump block through said outlet chamber; and, when said pump pin is pulled backward, the internal forces created in said pump diaphragm by stretching it will act to return said pump diaphragm to its minimum energy position, thereby increasing the volume of said pump chamber, reducing the pressure in said pump chamber below atmospheric pressure causing said inlet diaphragm to stretch into said inlet chamber, thereby allowing said liquid to be sucked out of said reservoir via said inlet chamber into said pump chamber.

2. A dosing pump according to claim 1, wherein, as the pump pin is moved forward, the pump diaphragm is stretched and its center is moved forward a distance that is greater than the diameter of said pump diaphragm.

3. A dosing pump according to claim 1, wherein the liquid is insulin.

4. A dosing pump according to claim 1, wherein, the output axle is coupled to the pump pin by means of an eccentric pin that is fixedly attached to said output axle and fits into a slot at the proximal end of said pump pin.

5. A dosing pump according to claim 1, wherein the output axle is coupled to the pump pin by means of a pinion gear fixedly attached to said output axle, wherein the teeth of said pinion gear mesh with the teeth of a rack gear that is an integral part of said pump pin.

6. A dosing pump according to claim 1, wherein the pump pin, motor, gear system, and the pump diaphragm are strong enough to enable to exert a force of at least five atmospheres on the liquid in the pump chamber when said pump pin pushes against said pump diaphragm.

7. A dosing pump according to claim 1, wherein, as the pump pin is pulled backwards, the energy stored in the pump diaphragm is released as said pump diaphragm returns to its unstretched state, thereby exerting a force of about two bars as said pump diaphragm contracts.

8. A dosing pump according to claim 1, comprising a pressure sensor diaphragm one side of which is in fluid communication with the outlet chamber when liquid flows through said outlet chamber; wherein when liquid is forced through said outlet chamber pressure, said pressure sensor diaphragm is caused to move relative to a pressure sensor, which measures parameters that can be translated into pressure measurements.

9. A dosing pump according to claim 8, wherein said pressure sensor is chosen from the following:
i) an optical sensor;
ii) a conductive silicone membrane, which changes electrical conductivity when stretched;
iii) a strain gauge buried in the pressure sensor; and
iv) an ultrasound sensor.

10. A dosing pump according to claim 8, wherein the output of the pressure sensor provides an indication that said dosing pump is operating properly and issues a real time warning if a problem with the flow of liquid from the reservoir to the body of the patient is detected.

11. A dosing pump according to claim 10, wherein the problem is one or more of the following:
i) a blockage in the fluid path from the reservoir to the body of the patient;
ii) a leak in said fluid path;
iii) air in said fluid path; or
iv) a non-constant supply of liquid from said reservoir.

12. A dosing pump according to claim 4, wherein the motor turns in one direction only and the gear system comprises a sensor to measure the exact instant when the direction of linear motion of the pump pin changes.

13. A dosing pump according to claim 5, wherein the direction in which the motor turns is reversed by the internal control unit based on the magnitude of the current drawn by said motor.

14. A dosing pump according to claim 1, wherein the gear system comprises a sensor to measure each rotation of the shaft of the motor.

15. A dosing pump according to claim 1, wherein said dosing pump is entirely disposable after the reservoir is emptied once.

16. A dosing pump according to claim 15, wherein the reservoir is a collapsible elastomeric bladder.

17. A dosing pump according to claim 15, wherein said dosing pump is attached directly to an infusion patch.

18. A dosing pump according to claim 17, comprising a quick release mechanism allowing said dosing pump to be easily temporarily disconnected from the infusion patch.

19. A dosing pump according to claim 1, comprising a button for administering a pre-determined bolus dose.

20. A dosing pump according to claim 1, wherein input/output to the internal control unit of said dosing pump is from a hand held remote control unit using an RFID or Bluetooth connection.

21. A dosing pump according to claim 20, wherein the hand held remote control unit is either a standard Palm-like device or a mobile phone.

22. A dosing pump according to claim 1, wherein said dosing pump is partially reusable.

23. A dosing pump according to claim 22, wherein the non-reusable parts of said dosing pump comprise the reservoir, the pump pin, the pump block, and optionally, depending on its location in said dosing pump, the sensor diaphragm.

24. A dosing pump according to claim 23, wherein the reservoir is a standard 3 ml insulin pen cartridge.

25. A dosing pump according to claim 1, wherein the reservoir is a collapsible bladder made of an elastomeric material.

26. A dosing pump according to claim 1, comprising an adhesive pad directly attached to the bottom surface of said pump for attaching said pump to the skin of a patient and a small hollow needle in liquid communication with the output channel of the pump block, which projects downward through said adhesive pad for penetrating the skin.

27. A dosing pump according to claim 22, wherein the non-reusable parts of said dosing pump are the pump block, the gear unit, the motor, the pressure sensor diaphragm, the reservoir, and the battery.
